(19) United States
(12) Patent Application Publication
(10) Pub. No.: US 2023/0126056 A1
(43) Pub. Date: Apr. 27, 2023

(54) ALLULOSE CONCENTRATES IN SOLID AMORPHOUS FORM

(71) Applicant: SAVANNA INGREDIENTS GMBH, Elsdorf (DE)

(72) Inventors: Anna JUNKLEWITZ, Wuppertal (DE); Stephan MOHR, Eushkirchen (DE); Steffen Butz, Kreuzau (DE); Michael BOGNAR, Dormagen (DE); Timo KOCH, Elsdorf (DE)

(21) Appl. No.: 17/798,549

(22) PCT Filed: Feb. 8, 2021
(86) PCT No.: PCT/EP2021/052966

§ 371 Date: Aug. 9, 2022

(30) Foreign Application Priority Data
Feb. 12, 2020 (EP) 20156797.1

(51) Int. Cl. C07H 3/02; C07H 1/06

(52) U.S. Cl. CPC C07H 3/02; C07H 1/06

(57) ABSTRACT

Proposed are allulose concentrates in solid amorphous form, which are characterized in that they contain at least approximately 20 wt. % allulose, and a method for producing said concentrates.

### FIELD OF INVENTION

The invention relates to the field of food technology and to allulose concentrates and a method for producing said concentrates.

### TECHNOLOGY BACKGROUND

Allulose (psicose) is a low-calorie sugar with a sweet taste similar to sugar. Allulose is one of many different sugars that occur in very small amounts in nature. Allulose was originally identified from wheat and has since been found in certain fruits such as jackfruit, figs and raisins. Allulose is found naturally in small amounts in a variety of sweet foods like caramel sauce, maple syrup, and brown sugar. Allulose is absorbed by the body but not metabolized, making it nearly calorie-free.

Due to the growing interest of a large part of the population in “healthy eating” and healthy living in general, allulose, as a zero-calorie sugar, has attracted a great deal of interest in the food industry and in the scientific community.

Typically, allulose is commercialized in the form of a syrup. However, these allulose syrups have considerable stability problems, so that the quality of the product is no longer optimal after prolonged storage, particularly with regard to sensory properties, color, purity and temperature stability.

Drying food is one of the oldest and at the same time most natural preservation methods and has proven to be a gentle way of preservation to this day.

Drying is generally understood to mean the removal of liquids from the material to be dried, through evaporation, vaporization or other technical methods. The dry material can be in the form of powder or granules, in pieces or slices or left whole, for example in the case of fruit. Due to the removal of moisture, the dry material has a significantly lower weight after the treatment, while at the same time the important and valuable components are retained.

Freeze drying is a particularly preferred drying method because it has proven to be a particularly gentle drying method in terms of taste or rehydration ability. With this method, however, the material to be dried must first be frozen in order to sublimate the water from it. The disadvantage of this method is that it is very energy-intensive.

### RELEVANT PRIOR ART

Various methods for the production of allulose are known from the prior art, for example from the documents WO 2018/087261 A1 (Pfeifer & Langen GmbH & Co. KG) or WO 2016/135358 A1 (Tate & Lyle Technology Limited).

### OBJECT OF THE INVENTION

The object of the present invention was therefore to provide allulose concentrates in solid, amorphous form, which are produced from an allulose syrup and are free from the aforementioned drawbacks. In particular, the novel allulose concentrates should have improved storage and temperature stability compared to allulose in syrup form, so that an allulose syrup is obtained after reconstitution by adding water, which is indistinguishable from the freshly prepared, original allulose syrup in terms of its composition and organoleptic properties.

At the same time, it was also an object of the present invention to provide an energy-saving method which enables the above-mentioned solid amorphous allulose concentrates to be produced.

### DESCRIPTION OF THE INVENTION

The subject of the present invention are allulose concentrates in solid amorphous form, which are characterized in that they have an allulose content of at least approximately 20 wt. %.

It has surprisingly been found that the allulose concentrates according to the invention have a significantly improved storage and temperature stability compared to the corresponding allulose in syrup form from which they were obtained.

Furthermore, it has surprisingly been found that after a storage period of more than one year, the solid amorphous allulose concentrates according to the invention allow the production of an allulose syrup by reconstitution by adding water, which was indistinguishable from the freshly prepared original allulose syrup from which it was obtained, in terms of its composition and organoleptic properties.

As used in the present invention, the term “concentrate” refers to dry or aqueous compositions containing a total amount of 20 to 99 wt. % allulose, based on total solids.

As used in the present invention, “syrup” is understood to mean a viscous, concentrated solution of a sugar or sugar substitute obtained by cooking or other techniques, in particular by enzymatic cleavage or conversion processes, from liquids or plant extracts containing sugar.

Allulose, also called psicose, is a ketohexose. For the purpose of the present invention, allulose is preferably provided in the form of the D enantiomer, i. e. D-allulose (CAS No. 551-68-8). D-Allulose can be present in the form of various anomers such as α-D-Allulose and β-D-Allulose.

In a preferred embodiment, the solid amorphous allulose concentrates according to the invention are present in amorphous form to an extent of at least 90 wt. %, preferably at least 95 wt. % and in particular from approximately 98 to approximately 99.5 wt. %.

In a preferred embodiment, the allulose concentrates according to the invention have an allulose content of at least approximately 40 wt. %, preferably at least approximately 50 wt. %, preferably at least approximately 60 wt. %, particularly preferably at least approximately 70 wt. %, and most preferably at least approximately 75 wt. %.

The allulose concentrates according to the invention of the present invention are characterized in that the allulose content is in the range from approximately 20 to approximately 99 wt. %.

In a preferred embodiment of the allulose concentrates according to the invention, the allulose content is in the range from approximately 25 to approximately 95 wt. %, preferably in the range from approximately 40 to approximately 90 wt. %, preferably in the range from approximately 45 to approximately 85 wt. %, and most preferably in the range of approximately 50% to approximately 80 wt. %.

In addition, the allulose concentrates according to the invention can also contain fructose. The allulose concentrates according to the invention can have a fructose content of a maximum of approximately 80 wt. %.

In a further preferred embodiment, the allulose concentrates according to the invention have a fructose content of a maximum of approximately 60 wt. %, preferably a maximum of approximately 50 wt. %, preferably a maximum of approximately 40 wt. %, particularly preferably a maximum of approximately 30 wt. %, and most preferably a maximum of approximately 25 wt. %.

In a preferred embodiment of the allulose concentrates according to the invention, the fructose content is in the range from approximately 25 to approximately 80 wt. %, preferably in the range from approximately 40 to approximately 60 wt. %, and particularly preferably in the range from approximately 45 to approximately 55 wt. %.

In a further preferred embodiment, the allulose concentrates according to the invention have an allulose content of at least approximately 20 wt. % and a maximum of approximately 80 wt. % fructose, preferably at least approximately 25 wt. % allulose and a maximum of approximately 75 wt. % fructose, preferably at least approximately 40 wt. % allulose and a maximum of approximately 60 wt. % fructose, preferably at least approximately 50 wt. % allulose and a maximum of approximately 50 wt. % fructose, preferably at least approximately 60 wt. % allulose and a maximum of approximately 40 wt. % fructose, preferably at least approximately 70 wt. % allulose and a maximum of approximately 30 wt. % fructose, and particularly preferably at least approximately 75 wt. % allulose and a maximum of approximately 25 wt. % fructose.

In a first preferred embodiment, the allulose:fructose weight ratio is approximately 20:80 to approximately 80:20, in a second embodiment approximately 90:10 to 95:5.

The solid amorphous allulose concentrates according to the invention are characterized in that they have a maximum water content of approximately 0.1 wt. %, preferably a maximum of approximately 0.05 wt. %, and particularly preferably a maximum of approximately 0.01 wt. %.

Another subject of the invention relates to a method for producing a solid amorphous allulose concentrate, comprising or consisting of the following steps:

(a) providing an allulose concentrate in syrup form,

(b) optionally concentrating the product from step (a),

(c) drying the intermediate product from step (b) or the allulose concentrate from step (a) on a spray, freeze or belt dryer with subsequent comminution, and finally

(d) discharging and, if necessary, subsequent packaging of the dry end product.

In the first part of the method, an allulose concentrate is initially provided in syrup form; it can be a commercially available allulose concentrate in syrup form or an allulose concentrate in syrup form which has been produced by a method from the prior art.

In a preferred embodiment, the allulose concentrate in syrup form from step (a) comprises or consists of the following components:

(i) from approximately 90% to approximately 95 wt. % allulose,

(ii) from approximately 0% to approximately 10 wt. % fructose,

(iii) 0 to approximately 5 wt. % other carbohydrates other than allulose and fructose, with the proviso that the quantities add up to 100 wt. %. For the sake of good order, it should be noted that embodiments which result in more or less than 100 wt. % are neither included in the invention nor covered by the claims. In any case, the person skilled in the art is able, on the basis of the description and examples, to find compositions that meet the technical teaching without having to be inventive.

In a preferred embodiment, the allulose concentrate in syrup form from step (a) has a water content of approximately 25 to approximately 40 wt. %, particularly preferably approximately 30 wt. %.

In another preferred embodiment, the allulose concentrate in syrup form from step (a) has a dry matter content of approximately 10% to approximately 30 wt. %.

At this point it should also be mentioned that for producing the solid amorphous allulose concentrates, which should have a purity of virtually 100% and be virtually free of fructose or other carbohydrates (other than allulose and fructose), it is advantageous if the allulose is crystallized from the allulose concentrate in syrup form from step (a).

Here, the allulose concentrate in syrup form from step (a) is subjected to evaporative, cooling or displacement crystallization with a protic solvent or a combination thereof.

The allulose crystals thus obtained are preferably filtered off and then subjected to drying with subsequent comminution; the dry end product thus obtained is then discharged (steps (c) and (d) of the method according to the invention).

Suitable protic solvents for the crystallization of the allulose from the allulose concentrate in syrup form are branched or unbranched C₁-C₆ alcohols, preferably ethanol and/or isopropanol. During the crystallization, the solid then consists entirely or at least predominantly of β-D-psicopyranose.

As already mentioned, the allulose concentrate in syrup form from step (a) can optionally be fed into an evaporator, preferably a rising plate film evaporator, in which it is subjected to a temperature gradient from approximately 100 to approximately 50° C. and in particular from approximately 95 to approximately 55° C. and/or a pressure gradient and gently dewatered, so that the intermediate product obtained in this way has a dry matter content of approximately 60 to approximately 85 wt. %, preferably approximately 80 wt. %. The allulose concentrate in syrup form from step (a) or the concentrated intermediate from step (b) is then also dried.

The method described above enables the production of the solid amorphous allulose concentrates. Therefore, a further subject of the present invention relates to amorphous solid allulose concentrates which are obtainable by

(a) providing an allulose concentrate in syrup form,

(b) optionally concentrating the concentrate from step (a),

(c) subjecting the intermediate of step (b) or the allulose concentrate of step (a) to drying on a spray, freeze or belt dryer followed by comminution, and finally

(d) discharging the dry end product and packaging it, if necessary.

With regard to the technical aspects of the production method, the above statements also apply, so that a repetition is unnecessary.

In summary, it can be stated that the solid amorphous allulose concentrates according to the invention represent an excellent alternative for storage over a longer period of time of typical allulose syrups, since the concentrates are characterized by improved resistance and stability.

In addition, allulose syrups can be reproducibly produced from the solid amorphous allulose concentrates according to the invention in a very simple and efficient manner, as demonstrated in the example of the present invention.

### EXAMPLES

The present invention will be more readily understood with reference to the following examples. However, these examples are only intended to illustrate the invention and should not be construed as limiting the scope of the invention.

### Example 1

A freshly prepared allulose concentrate in syrup form (Product A) with a dry matter content of 70 wt. % (65 wt. % allulose and 5 wt. % fructose) was evaporated in a rising plate film evaporator by subjecting it to a temperature gradient of 95 to 55° C. until it reached a dry matter content of 75 wt. %. Using a feed pump, the intermediate product obtained was placed on the conveyor belt of a vacuum belt dryer. Said vacuum belt dryer had three heating zones (60, 50 and 40° C.) and one cooling zone (20° C.) (pressure: 40 mbar). The product obtained was comminuted and packaged, and stored at 22° C. for 14 months. After this time, the syrup was reconstituted by the addition of an appropriate amount of water to give an allulose concentrate in syrup form (Product A1), which was identical to the original product A in terms of composition.

### Comparative Example V1

A glass bottle was filled with a freshly prepared Product A, as defined in Example 1. The product was stored for 14 months under identical conditions as the product according to the invention according to Example 1, so that a Product A2 was obtained.

### Evaluation of the Products A, A1 and A2

The freshly prepared syrup (Product A), the syrup prepared freshly from the allulose concentrate according to the invention (after 14 months of storage) (Product A1) and the Product A2 (product A after 14 months of storage) were evaluated by a panel consisting of five experienced and trained testers (3=pronounced, 2=present, 1=not ascertainable). The following parameters were evaluated:

optics (tanning)

taste (bitter and metallic impression)

The results are summarized in Table 1.

TABLE 1
Product rating

| Products | optics | taste |
|---|---|---|
| Product A | 1 | 1 |
| Product A1 | 1 | 1 |
| Product A2 | 3 | 3 |

The data in Table 1 clearly show that storing the solid amorphous allulose concentrates according to the invention for 14 months has no effect on the quality of the syrup produced therefrom. The concentrates according to the invention show an excellent solubility, so that a syrup can be readily reconstituted by adding an appropriate amount of water at room temperature and by means of usual stirring.

On the other hand, due to the instability of the syrup, Product A2 showed a significant deterioration in both the optical and the organoleptic properties,

The solid amorphous allulose concentrates according to the invention therefore represent an excellent alternative for storage over a longer period of time of typical allulose syrups, since the concentrates are characterized by their resistance and stability. In addition, allulose syrups can be reproducibly produced from the solid amorphous allulose concentrates according to the invention in a very simple and efficient manner.

1. An allulose concentrate in solid amorphous form, characterized in that the concentrate has an allulose content of at least approximately 20 wt. %.

2. The allulose concentrate according to claim 1, characterized in that the concentrate has a maximum fructose content of approximately 80 wt. %.

3. The allulose concentrate according to claims 1 and/or 2, characterized in that the concentrate has an allulose content of at least approximately 80 wt. % and a maximum of approximately 20 wt. % fructose.

4. The allulose concentrate according to at least one of the preceding claims 1 to 3, characterized in that the weight ratio of allulose:fructose is approximately 20:80 to approximately 80:20.

5. The allulose concentrate according to at least one of the preceding claims 1 to 4, characterized in that the concentrate has a maximum water content of approximately 0.1 wt. %.

6. A method for producing an allulose concentrate in solid amorphous form, comprising or consisting of the following steps:
(a) providing an allulose concentrate in syrup form,
(b) concentrating the product from step (a),
(c) drying the intermediate product from step (b) on a spray, freeze or belt dryer with subsequent comminution, and finally
(d) discharging and, if necessary, subsequent packaging of the dry end product under vacuum.

7. The method according to claim 6, characterized in that the allulose concentrate in syrup form from step (a) comprises or consists of the following components:
(i) from approximately 90 to approximately 95 wt. % allulose,
(ii) from approximately 5 to approximately 10 wt. % fructose,
(iii) from 0 to approximately 5 wt. % other carbohydrates other than allulose and fructose,
with the proviso that the quantities add up to 100 wt. %.

8. The method according to at least one of claims 6 and/or 7, characterized in that the allulose concentrate in syrup form from step (a) has a water content of approximately 5 to approximately 40 wt. %.

9. The method according to at least one of claims 6 to 8, characterized in that the allulose concentrate in syrup form from step (a) has a water content of approximately 25 to approximately 30 wt. %.

10. The method according to at least one of claims 6 to 9, characterized in that the allulose concentrate in syrup form from step (a) has a dry matter content of approximately 10 to approximately 30 wt. %.

11. The method according to at least one of claims 6 to 10, characterized in that the intermediate product from step (b) has a dry matter content of approximately 40 to approximately 80 wt. %.

12. The method according to at least one of claims 6 to 11, characterized in that the intermediate product from step (b) has a dry matter content of approximately 75 wt. %.

13. The method according to at least one of claims 6 to 12, characterized in that drying is carried out at temperatures in the range from approximately 60 to approximately 130° C. and at a pressure of approximately 15 to approximately 40 mbar.
